(19) United States
(12) Patent Application Publication
(10) Pub. No.: US 2006/0270634 A1
(43) Pub. Date: Nov. 30, 2006

(54) Acetal phosphate-derived LPA mimics, PPARgamma activators, and autotaxin inhibitors

(76) Inventors: Duane D. Miller, Memphis, TN (US); Gabor J. Tigyi, Memphis, TN (US); Veeresa Gududuru, Memphis, TN (US); Yuko Fujiwara, Memphis, TN (US); Daniel Baker, Memphis, TN (US); Michelle D. Walker, Alexandria, VA (US); Gangadar Good Durgam, Memphis, TN (US)

Correspondence Address: DONNA J. RUSSELL, 1492 ANTHONY WAY, MT. JULIET, TN, 37122, US

(21) Appl. No.: 11/418,561

(22) Filed: May 6, 2006

Related U.S. Application Data
(60) Provisional application No. 60/678,498, filed on May 6, 2005.
(60) Provisional application No. 60/705,556, filed on Aug. 4, 2005.

(51) Int. Cl. A61K 31/665

(52) U.S. Cl. 514/99; 549/216

(57) ABSTRACT

Disclosed are compositions that modulate the effects of extracellular LPA receptors, the intracellular PPARγ receptor, and autotaxin, and methods for their use.

### CROSS-REFERENCE TO RELATED APPLICATIONS

This application claims the benefit of priority of earlier-filed U.S. provisional patent applications No. 60/678,498 filed May 6, 2005 and 60/705,556 filed Aug. 4, 2005.

### STATEMENT OF GOVERNMENT RIGHTS

The present invention was made, at least in part, with funding received from the National Institutes of Health under grants CA92160 and HL61469, and from the Department of Defense under grant DMAD 17-01-0830. The U.S. government may retain certain rights in this invention.

### FIELD OF THE INVENTION

The present invention relates to the use of lysophosphatidic acid (LPA) analogs to modulate LPA receptor (LPA-R) activity, peroxisome proliferator-activated receptor gamma (PPARγ) activity, and to inhibit lysophospholipase D (autotaxin, ATX) activity.

### BACKGROUND OF THE INVENTION

Lysophosphatidic acid (LPA; 1-acyl-3-phosphoglycerol) interacts with both intracellular and extracellular targets. Included among the known targets of LPA are the cell-surface G-protein coupled receptors (GPCR), the nuclear peroxisome-proliferator-activated receptor γ (PPARγ), and the secreted cancer cell motility factor autotaxin.

Not surprisingly, given the number of targets with which LPA has thus far been found to interact, it has also been demonstrated to play a role in a variety of physiological pathways that have also been associated with cellular and tissue development, as well as pathways associated with disease states such as atherosclerosis, cancer, diabetes, and even acne. There are, therefore, numerous efforts being made in laboratories around the world to determine how best to modulate LPA in a more target-specific manner, as well as to develop agents that modulate LPA and/or the targets of LPA for therapeutic use. One such agent is the PPAR-γ agonist rosiglitazone maleate (5-((4-(2-(methyl-2-pyridinylamino)ethoxy)phenyl)methyl)-2,4-thiazolidinedione, which is currently produced by Glaxo Smith Kline under the brand name Avandia®. Rosiglitazone is an oral drug used for treating patients with type 2 diabetes.

Given the importance of LPA and its associated targets in metabolism, there is a need in the art for new agents that may selectively modulate the effects of these molecules and be of therapeutic value in the treatment and prevention of disease.

### SUMMARY OF THE INVENTION

The present invention relates to LPA analogs that act as agonists or antagonists of LPA₁, LPA₁, LPA₃, PPARγ, and/or autotaxin, the LPA analogs comprising acetal phosphatidic acids. Acetal phosphatidic acid LPA analogs as described herein may be described by the formula 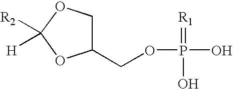
where R₁ is O or S, and R₂ is a straight or branched chain saturated or unsaturated, linear or cyclic hydrocarbon, substituted or unsubstituted, preferably C6 to C24, having an agonist or antagonist effect on LPA₁, LPA₂, LPA₃, PPARγ, and/or autotaxin. In some embodiments, the analogs may comprise water soluble salts of an acetal phosphatidic acid.

The invention also provides a therapeutic method of modulating an LPA-, PPARγ-, and/or autotaxin-mediated disease in a human or animal subject, the method comprising administering to the subject a therapeutically effective amount of at least one acetal phosphatidic acid having agonist or antagonist activity on LPA₁, LPA₁, LPA₃, PPARγ, autotaxin, or a combination thereof.

Also provided are fatty acid alcohol derivatives having LPA receptor agonist/antagonist activity, PPARγ agonist/antagonist activity, and autotaxin modulating activity, as well as a method of use of such fatty acid alcohol LPA analogs to treat or prevent LPA-, PPARγ, and/or autotaxin-mediated disease.

### BRIEF DESCRIPTION OF THE DRAWINGS

FIG. 1 illustrates the chemical structures of LPA (lysophosphatidic acid) and Darmstoff, where 1, 2, and 3 represent the predominant acetal phosphatidic acids comprising Darmstoff.

FIGS. 2-4 illustrate the chemical synthesis schemes utilized to synthesize Darmstoff analogs comprising modulators of LPA-R, PPARγ, and ATX as described in the present invention disclosure.

FIGS. 5-7 illustrate the chemical synthesis schemes utilized to synthesize phosphatidic acid 8:0 derivatives described herein.

FIG. 8 graphs dose-response relationships for LPA 18:1, 12b and 13b in RH7777 cells expressing LPA, (FIG. 8a) and LPA₃ (FIG. 8b). Intracellular Ca²⁺ transients were measured in response to the application of increasing concentrations of compounds 12b and 13b and compared to transients elicited by LPA 18:1. Data points represent the average of four measurements. (2R) Alkyl PA analogs (12b and 13b) are agonists at LPA₁ and LPA₃ receptors expressed in RH7777 cells.

FIG. 9 graphs results of in vitro PPARγ activation by PA analogs in CV1 cells transfected with PPARγ and PPRE-Acox-Rluc reporter gene, comparing the effects with the Rosiglitazone, a known PPARγ agonist. CV1 cells were treated with vehicle or 10 μM of test compound dissolved in DMSO for 20 h. Luciferase and β-galactosidase activities (mean±SEM) were measured in the cell lysate (n=4). * P<0.05, significant differences over vehicle control.

### DETAILED DESCRIPTION

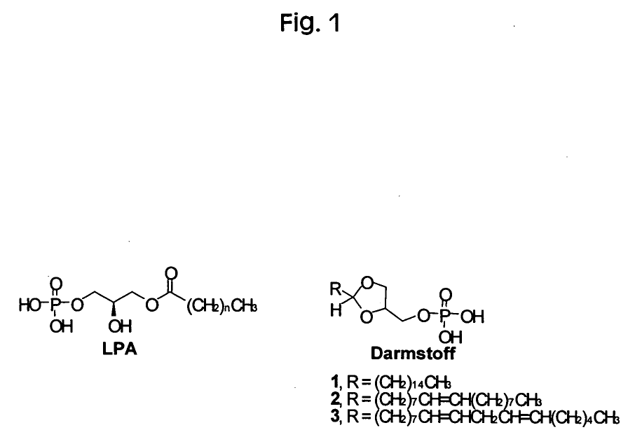

In 1949 Vogt reported isolation of an acidic phospholipid from the horse intestine that was capable of effecting smooth muscle contraction. This substance named Darmstoff at that time by Vogt has been shown to be a mixture of acetal phosphatidic acids. The inventors have demonstrated that Darmstoff analogs constitute a new class of subtype-selective LPA agonists and antagonists and developed a general and facile method for the synthesis of water-soluble salts of these analogs. They have demonstrated that Darmstoff analogs provide subtype-specific LPA G-protein coupled receptor (GPCR) ligands, as well as activators of nuclear transcription factor PPARγ and inhibitors of lysophospholipase D (autotaxin). The structures of LPA and the three major acetal phosphatidic acids which generally comprise Darmstoff are shown in FIG. 1.

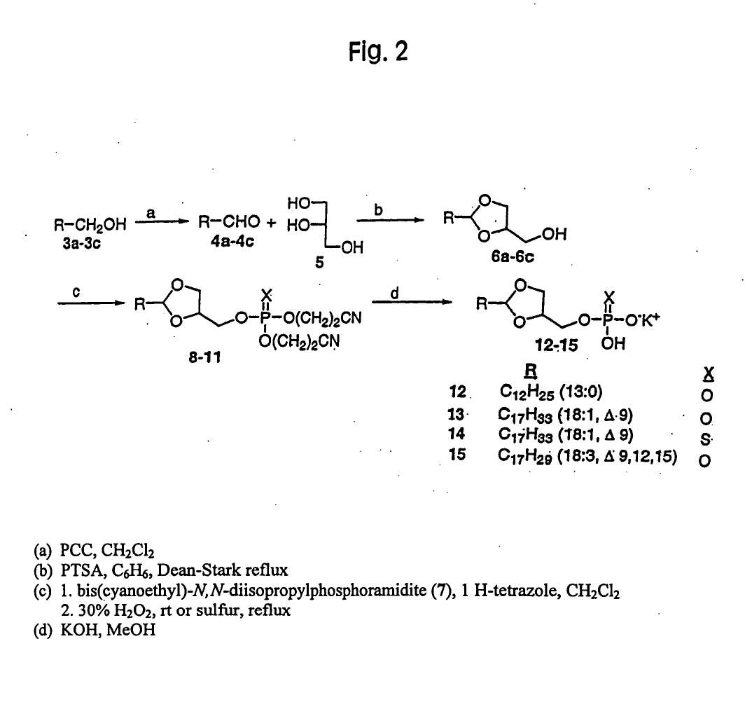

Pyridinium chloride (PCC) mediated oxidation of fatty alcohols produced the corresponding aldehydes, which were condensed with glycerol in the presence of PTSA under conditions previously reported in the literature to give dioxolanes (FIG. 2). Phoshorylation of dioxolanes using bis(cyanoethyl)-N,N-diisopropylphosphoramidite 7 in the presence of 1H-tetrazole formed phosphorous acid esters that were converted in situ to phosphate or thiophosphate esters using hydrogen peroxide or sulfur, respectively. Finally, treatment of the phosphate or thiophosphate esters with methanolic KOH at ambient temperature provided potassium salts of Darmstoff analogs.

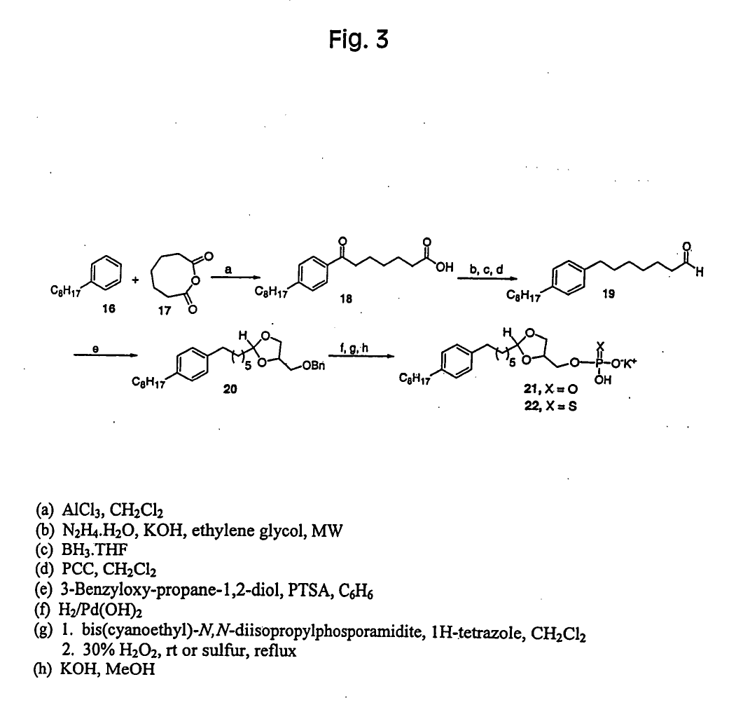

The synthesis of compounds 21 and 22 containing a phenyl ring in the lipid chain is shown in FIG. 3. Friedel-Crafts acylation of n-octyl benzene with pimelic anhydride gave arylketo acid 18 that was converted to the required aldehyde 19 in three steps. Condensation of 19 with 3-benzyloxy-propane-1,2-diol under standard conditions formed the dioxolane 20. Debenzylation of 20 followed by phosphorylation and removal of the protecting groups gave target compounds 21 and 22.

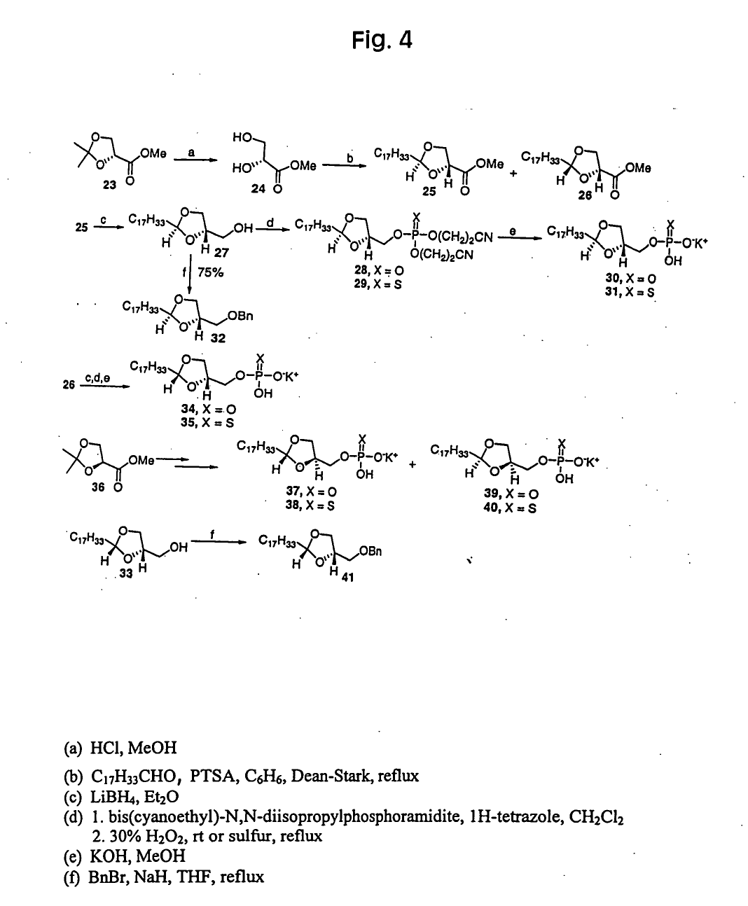

To investigate the effect of stereochemistry on biological activity, the inventors synthesized all stereoisomers of Darmstoff analogs 13 and 14. 2,4-disubstituted-1,3-dioxolanes of this type exist as a mixture of four stereoisomers. The inventors' synthetic approach for the preparation of the four possible stereoisomers of 13 and 14 is outlined in FIG. 4. Accordingly, acid mediated removal of the isopropylidene group from commercially available 23 (R-isomer) gave methyl glycerate 24 in a quantitative yield. The acid-catalyzed condensation of cis-9-octadecenal with 24 afforded a mixture of dioxolanes 25 and 26, which were readily separated by column chromatography. LiBH₄ mediated reduction of the ester functionality of 25 gave alcohol 27 that was phosphorylated using 7 to form phosphate 28 and thiophosphate 29. Finally, treatment of these esters with methanolic KOH gave the corresponding Darmstoff stereoisomers 30 and 31 as shown in FIG. 4. Similarly, dioxolane intermediate 26 was converted to target compounds 34 and 35 using the same chemistry. Synthesis of the other four Darmstoff stereoisomers 37-40 was performed using the same procedure, but used 36 (the S-isomer of 23) as the starting material. To examine the purity of these stereoisomers, HPLC profiles of compounds 32 and 41 were analyzed. Benzyl ethers (32 and 41, FIG. 4) were prepared to increase their detection by UV. HPLC analysis (Chiralpak AS-RH 150×4.6 m, 1:1 water-acetonitrile) of benzyl ethers confirmed the purity of 32 and 41. All compounds were fully characterized spectrophotometrically.

The biological effects of all synthesized compounds were testing using three high-throughput assays. Representative compounds are shown in Tables 1 and 2. First, intracellular calcium transients in rat hepatoma (RH777, an LPA receptor null cell) cell lines individually expressing either LPA₁, LPA₂, and LPA₃ receptors were analyzed to evaluate compounds as agonists or antagonists. Wild type RH7777 cells did not respond to any of the Darmstoff analogs. Second, PPARγ activation was examined in CV1 cells (an African green monkey kidney cell line), transfected with an acyl-coenzyme A oxidase-luciferase (PPRE-Acox-Rluc) reporter gene construct according to a protocol similar to that described by McIntyre, et al. (Proc. Natl. Acad. Sci. USA. 100:131-136 (2003)). The PPRE-Acox-Rluc construct contains a renilla luciferase coding region, an acyl-CoA oxidase coding region, and a PPAR response element coding region. Third, inhibition of the lysophospholipase D autotoxin was determined using a previously described procedure.

Compound 12 (Table 1) containing a C13 alkyl chain and no double bond inhibited Ca²⁺ mobilization in cells expressing all three LPA GPCRs, thereby providing a pantagonist of LPA₁₋₃. An increase in chain length to C18 and introduction of the C₉═C₁₀ double bond resulted in analog 13, which produced LPA_{1/3} antagonist activity. Oleoyl-LPA is an agonist of LPA₁₋₃ while Darmstoff analog 13 containing an oleoyl chain at the C-2 position of the 1,3-dioxolane inhibited LPA₁₋₃ receptors, indicating that the acetal moiety plays a significant role in ligand recognition. To examine the effect of this modification with the Darmstoff series, compound 14 was synthesized. This analog was an agonist at all three LPA receptor subtypes and was most potent at LPA₃ (EC₅₀ of 639 nM). The phosphate analog 15 with conjugated double bonds at C₉═C₁₀, C₁₂═C₁₃, and C₁₅═C₁₆ positions was an agonist for all three LPA receptors. Though analog 15 was less potent than 14, these compounds were identified as two LPA GPCR pan-agonists.

A multitude of aldehydes are produced via oxidative cleavage of unsaturated fatty acids and their phospholipid derivatives. The cis-olefinic bond of analogs 13-15 is susceptible to oxidative cleavage. In order to avoid this problem and to examine the effect of structural rigidity on biological activity, the inventors replaced the double bond with an aromatic ring and screened against LPA GPCR, PPARγ, and autotoxin. Incorporation of an aromatic ring in the alkyl chain gave compounds 21 and 22. Analog 21 was an antagonist of LPA_{1/3} receptors but had no effect against LPA₂. The thiophosphate analog, compound 22 was a weak LPA, antagonist, without any effect on LPA₂ but stimulated LPA₃ with an EC₅₀ of 692 nM (Eₘₐₓ=87%).

To examine the importance of stereochemistry on biological activity, the inventors analyzed pure stereoisomers (Table 3) with respect to LPA GPCR activation. Results indicated that, regardless of stereochemistry at C-2 and C-4, Darmstoff analogs 30, 34, 37, and 39 with phosphate head groups were LPA₃ antagonists, whereas analogs with thiophosphate groups 31, 35, 38 and 40 were pan-agonists. Among the phosphate stereoisomers, analog 34 was identified as the most potent LPA₃ antagonist with an IC₅₀ of 136 nM (Ki=83 nM). Interestingly, compound 30 weakly activated LPA_{1/3} and was a partial LPA₂ agonist with an EC₅₀ of 1.17 μM (Eₘₐₓ=39%). Stereoisomers with a thiophosphate head group were found to be more potent than parent compound 14. In this series, all other stereoisomers (31, 35 and 38) with the exception of 40 were full agonists of LPA₃ receptor, with the most potent being 31 (EC₅₀ of 127 nM, Eₘₐₓ=127%).

PPARγ is a lipid-activated transcription factor that belongs to the nuclear hormone superfamily. The inventors examined the activity of all synthesized Darmstoff analogs as PPARγ activators in vitro in CV1 cells using a PPRE-Acox-Rluc reporter gene assay. Rosiglitazone, a known PPARγ agonist, was used as a positive control for comparison. The results shown in Table 3 indicate that all tested Darmstoff analogs, regardless of whether they had been found to be antagonists or agonists of LPA GPCR, activated the PPARγ reporter construct.

LPA is liberated as the product of lysophosphatidylcholine hyrolysis by the lysophospholipase D, autotaxin (ATX). The inventors screened Darmstoff analogs for ATX inhibition. The IC₅₀ values and percentage of inhibition for Darmstoff analogs are listed in Table 3. As indicated by the data, all tested analogs are capable of ATX inhibition independently of their ligand properties at LPA GPCR and PPARγ. Of the tested compounds, 31, an LPA₁₋₃ pan-agonist with preference for LPA₃ was the most effective ATX inhibitor, with an IC₅₀ of 252 nM.

Synthesized compounds were tested for induction and inhibition of LPA-induced calcium transients in rat hepatoma (RH7777) cell lines that stably express individual LPA₁, LPA₂, and LPA₃ receptors as described in the literature using a FlexStation II automated fluorimeter (Molecular Devices, Sunnyvale, Calif.). The results are shown in Table 1. Compound 4 inhibited LPA response with an IC₅₀ of 1.1 μM and 2.87 μM for LPA₁ and LPA₃ respectively. However, the same compound was a weak agonist of LPA₂ with an EC₅₀ of 1.18 μM (Table 1). Interestingly, compound 5 was a full LPA₂ agonist, and LPA_{1/3} antagonist. Modifications of the head group and double bond in compound 5 to analogs 6 and 7 respectively, provided two new pan-agonists of LPA_{1/2/3}. Replacement of the cis-olefinic bond in 6 (pan-agonist of LPA_{1/2/3}) with an aromatic ring provided a sub-type selective LPA₃ antagonist (15) which has no effect on LPA_{1/2}.

TABLE 1

| | | LPA₁ (nM) | | LPA₂ (nM) | | LPA₃ (nM) | |
|---|---|---|---|---|---|---|---|
| ID | Structure | EC₅₀ | IC₅₀ | EC₅₀ | IC₅₀ | EC₅₀ | IC₅₀ |
| 4 | 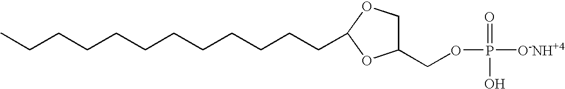 | NE | 1110 | 1180 | NE | NE | 2870 |
| 5 | 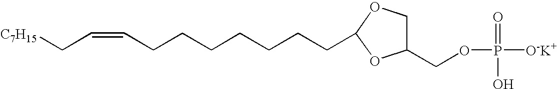 | NE | 915 | 68 | NE | NE | 527 |
| 6 | 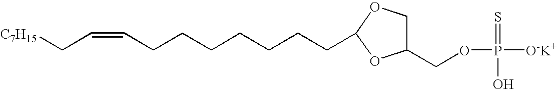 | 981 | NE | 34 | NE | 639 | NE |
| 7 | 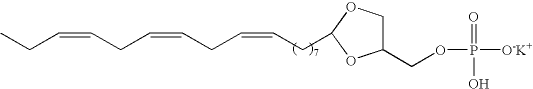 | 3598 | NE | 105 | NE | 7590 | NE |
| 14 | 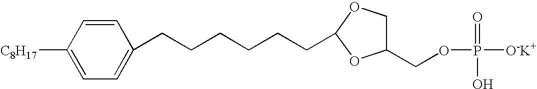 | NE | 4660 | NE | NE | 692 | NE |
| 15 | 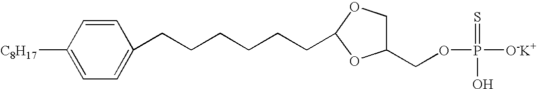 | NE | NE | NE | NE | NE | 504 |
| 21 | 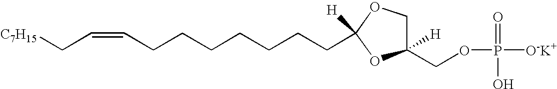 | ND | ND | >10000 | NE | NE | 484 |
| 22 | 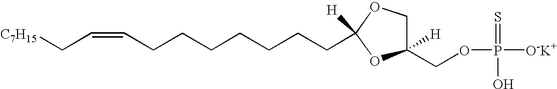 | ND | ND | 1540 | NE | 204 | NE |
| 23 | 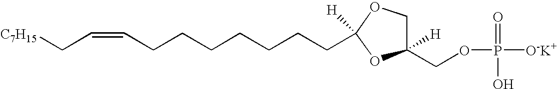 | ND | ND | NE | NE | ND | ND |
| 24 | 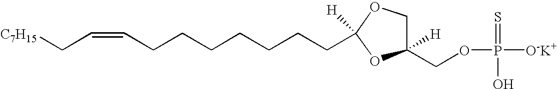 | ND | ND | 1320 | NE | ND | ND |
| 25 | 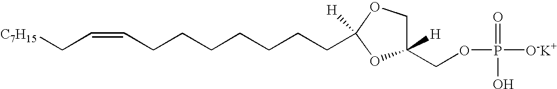 | ND | ND | 1170 | NE | ND | ND |
| 26 | 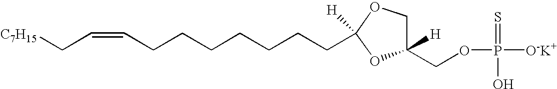 | ND | ND | ND | ND | ND | ND |
| 27 | 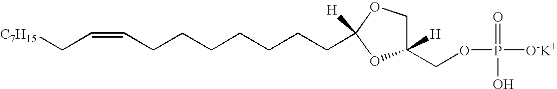 | ND | ND | ND | ND | ND | ND |
| 28 | 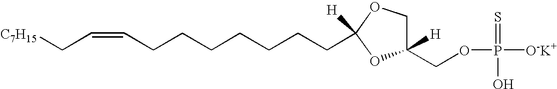 | ND | ND | 1710 | NE | ND | ND |

NE = No Effect, ND = Not Determined

TABLE 2

| Compound | LPA1 | LPA2 | LPA3 |
|---|---|---|---|
| 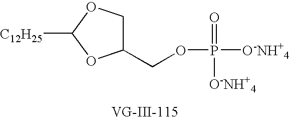 | IC₅₀: 1.11 uM (200 nM LPA) IC₅₀: 2.15 uM (300 nM LPA) | EC₅₀: 1.18 uM (weak agonist) *transient | IC₅₀: 2.87 uM (200 nM LPA) |
| 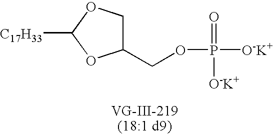 | IC₅₀: 915 nM (200 nM LPA) IC₅₀: 1.24 uM (300 nM LPA) | EC₅₀: 68 nM (full agonist) *transient | IC₅₀: 527 nM (200 nM LPA) |
| 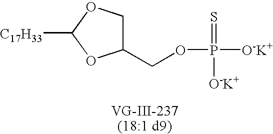 | EC₅₀: 981 nM (Partial agonist) | EC₅₀: 34 nM (almost full agonist) *transient | EC₅₀: 639 nM (Partial agonist) |
| 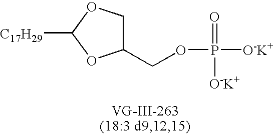 | EC₅₀: 3.598 uM (Partial agonist) | EC₅₀: 105 nM (full agonist) *transient | EC₅₀: 7.59 uM (Partial agonist) |
| 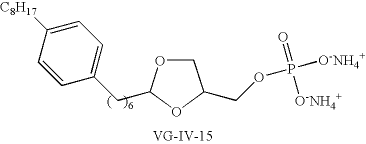 | IC₅₀: 4.66 uM (200 nM LPA) | | EC₅₀: 0.692 uM (Partial agonist) |
| 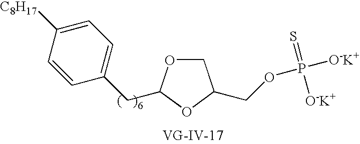 | Weak antagonist | | IC₅₀: 0.504 uM (200 nM LPA) |

TABLE 3

| | LPA₁ EC₅₀ | LPA₁ IC₅₀ | LPA₂ EC₅₀ | LPA₂ IC₅₀ | LPA₃ EC₅₀ | LPA₃ IC₅₀ | | ATX IC₅₀ |
|---|---|---|---|---|---|---|---|---|
| Analog | (Eₘₐₓ)^{a} nm | (Kᵢ) nm | (Eₘₐₓ)^{a} nm | (Kᵢ) nm | (Eₘₐₓ)^{a} nm | (Kᵢ) nm | PPARγ | (% inhib. nM) |
| 12 | NE^{b} | 1110 (652) | NE | 7430 (745) | NE | 2870 (681) | Agonist | 232 (26) |
| 13 | NE | 915 (497) | >10000 | NE | NE | 527 (548) | Agonist | 141 (30) |
| 14 | 981 (45) | NE | 1170 (87) | NE | 639 (73) | NE | Agonist | 415 (51) |
| 15 | 3600 (55) | NE | 1710 (51) | NE | 7590 (29) | NE | Agonist | 803 (54) |
| 21 | NE | 4660 (1930) | NE | NE | NE | 504 (171) | Agonist | 106 (10) |
| 22 | NE | WA^{c} | NE | NE | 692 (87) | NE | Agonist | 449 (55) |
| 30 | NE | WA | 1170 (39) | NE | NE | WA | Agonist | 120 (30) |
| 31 | 1580 (89) | NE | 1300 (77) | NE | 127 (127) | NE | Agonist | 252 (74) |
| 34 | NE | NE | 1710 (42) | NE | 136 (83) | NE | Agonist | 97 (28) |
| 35 | 1410 (71) | NE | 1090 (85) | NE | 194 (113) | NE | Agonist | 344 (66) |
| 37 | >10000 | NE | >10000 | NE | NE | 484 (241) | Agonist | 238 (46) |
| 38 | 2260 (68) | NE | 1540 (72) | NE | 204 (102) | NE | Agonist | 363 (64) |
| 39 | NE | WA | NE | NE | NE | 209 (77) | Agonist | 178 (25) |
| 40 | 1560 (65) | NE | 1320 (87) | NE | 265 (78) | NE | Agonist | 403 (60) |

^{a}Eₘₐₓ = maximal efficacy of drug/maximal efficacy of LPA 18:1, expressed as a percentage.
^{b}NE = no effect observed at the highest concentration (30 μM) tested.
^{c}WA = weak antagonist.

The invention therefore provides LPA receptor agonists and antagonists, as PPARγ agonists having the structure: 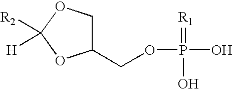
where R₁ is O or S, and R₂ is a straight or branched chain saturated or unsaturated, linear or cyclic hydrocarbon, substituted or unsubstituted, preferably C6 to C24, having an agonist or antagonist effect on LPA₁, LPA₂, LPA₃, PPARγ, and/or autotaxin. Compounds described by the present invention may also include, for example, salts, preferably water-soluble salts, such as ammonium, diammonium, potassium salts of the acetal phosphatidic acids.

The invention also provides methods for treating LPA- and/or PPARγ-mediated diseases by administering to a patient a therapeutically effective amount of an acetal phosphatidic acid analog of lysophosphatidic acid. A compound of the present invention may be provided, for example, as a therapeutic agent for the treatment and/or prevention of atherosclerosis, diabetes, cancer, and other LPA- and/or PPARγ-mediated diseases.

Modified alkyl-phenyl-alkyl phosphoric acid esters and straight chain di-halo phosphonates were synthesized by methods described in U.S. patent application Ser. No. 10/963,085 (Publication No. 2006/0009507A1) and tested for activity against the LPA receptors. A preferred scheme for the synthesis of the difluoro-alkyl phosphonates and reagents (a) (i) LDA, −78° C., THF; (ii) C₁₄H₂₉Br, 40% (b) (i) TMSBr, CH₂Cl_{2,} 6 h., rt; (b) MeOH/H₂O, 78% is shown below and effects of synthesized compounds on LPA receptor subtypes are shown in Table 4.

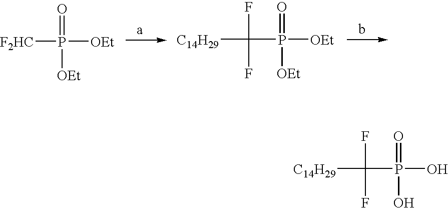

TABLE 4

| Compound | LPA1 | LPA2 | LPA3 |
|---|---|---|---|
| 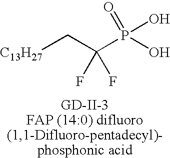 | Weak inhibition | EC₅₀:˜10 uM (40%) Weak agonist | IC₅₀: 1.51 uM (200 nM LPA) |
| 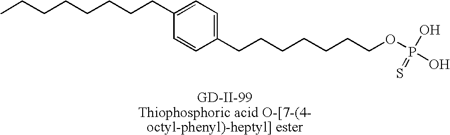 | EC₅₀: 4.83 uM (Partial agonist) | EC₅₀: 6.06 uM (full agonist) | EC₅₀: 0.858 uM (Partial agonist) |
| 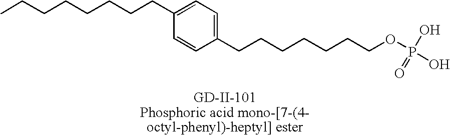 | EC₅₀: 21.5 uM (Partial agonist) | Weak antagonist | IC₅₀: 1.76 uM (200 nM LPA) |

Based on the observation that the shorter chain LPAs exert little or no activity on LPA receptors, the inventors identified short chain phosphatidic acid derivatives dioctanoyl glycerol pyrophosphate (DGPP 8:0, 1) and phosphatidic acid 8:0 (PA 8:0, 2) as subtype-selective LPA₁ and LPA₃ receptor antagonists.

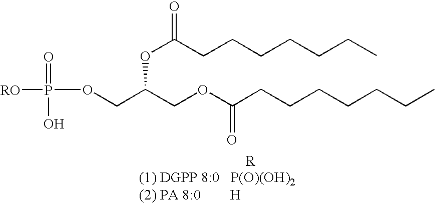

The inventors had discovered that the replacement of phosphate headgroup by thiophosphate, in fatty alcohol phosphates (FAP) series, had a positive effect by improving the agonist as well as antagonist activities at LPA GPCR. To develop improved agents for LPA-R binding, they synthesized stereoisomers of PA 8:0 analogs evaluated their interaction with LPA GPCR and PPARγ. Their data indicated that LPA receptors stereoselectively interact with the glycerol backbone modified ligands. With dioctyl PA 8:0 compounds, they observed stereospecific responses, in which (R)-isomers found to be agonists whereas the (S)-isomers were antagonists of LPA GPCR. From this series, they identified compound 13b as a potent LPA₃ receptor subtype-selective agonist (EC₅₀=3 nM), and 8b as a potent and selective LPA₃ receptor agonist (Kᵢ=5 nM). Serinde diamide phosphate 19b was identified as an LPA₃ receptor specific antagonist with no effect on LPA₁, LPA₂ and PPARγ.

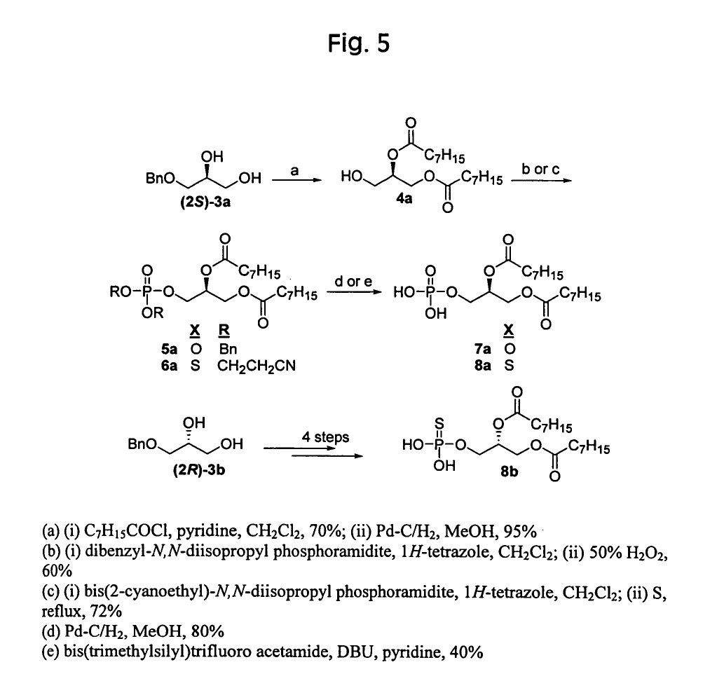

Dioctanoyl PA analogs were synthesized as shown in FIG. 5. Commercially available (2S)-3-benzyloxy-1,2-propanediol (3a) was diacylated with octanoylchloride followed by debenzylation under catalytic hydrogenation conditions provided the alcohol (4a). The alcohol (4a) was then phosphorylated using dibenzyl-N,N-diisopropyl phosphoramidite to yield dibenzyl protected phosphate (5a), which upon catalytic hydrogenation afforded the corresponding (2S)-dioctanoyl PA compound (7a). Treatment of 4a with bis(2-cyanoethyl)-N,N-diisopropyl phosphoramidite followed by reflux in presence of elemental sulfur provided the dicyanoethyl protected thiophosphate (8a). The target thiophosphatidic acid 8:0 (TPA 8:0, 8a) was obtained by deprotection of cyanoethyl groups using bis(trimethylsilyl)trifluoro acetamide and pyridine. Similarly, the (2R)-thiophosphate analog 8b was synthesized from (2R)-3-benzyloxy-1,2-propanediol in 4 steps. The (2R)-dioctanoyl PA compound (2) used in this study was purchased from commercial sources.

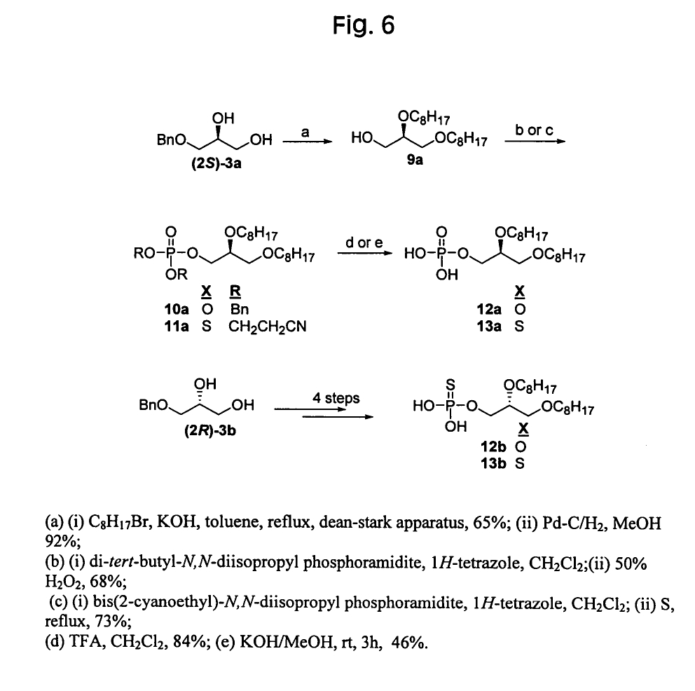

The dialkyl PA 8:0 (APA 8:0) analogs were synthesized as shown in FIG. 6. Alkylation of commercially available (2S)-3-benzyloxy-1,2-propanediol (3a) with octylbromide followed by debenzylation provided the alcohol (9a). The alcohol (9a) was then phosphorylated using phosphoramidite chemistry to di-tert-butyl protected phosphate (10a), which, upon treatment with TFA, gave the corresponding (2S)-dioctyl PA compound (12a). Treatment of 9a with bis(2-cyanoethyl)-N,N-diisopropyl phosphoramidite followed by reflux in presence of elemental sulfur provided the di-cyanoethyl protected thiophosphate (11a). The deprotection of cyanoethyl groups under basic conditions with treatment of KOH in methanol furnished the target dialkyl thiophosphatidic acid 8:0 (ATPA 8:0) compound 13a. Similarly, the (2R)-analogs 12b and 13b were synthesized from (2R)-3-benzyloxy-1,2-propanediol (8b) in 4 steps.

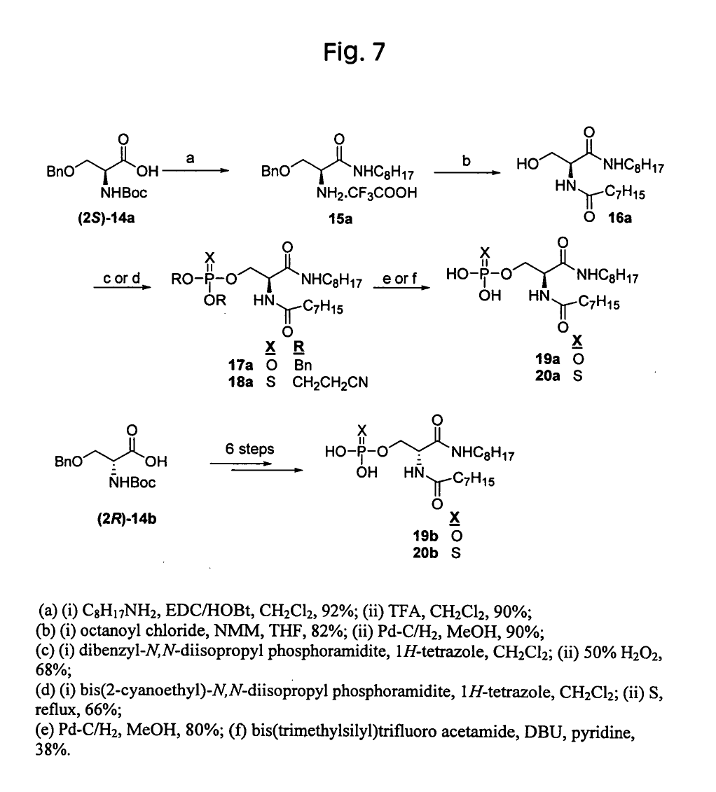

The serinediamide phosphate/thiophosphate (SDP/SDTP) analogs were synthesized as outlined in FIG. 7. O-benzyl-Boc-(L)-serine (14a) was coupled with octylamine using EDC and HOBt, and deprotection with TFA gave compound 15a. 15a was acylated using octanoyl chloride followed by debenzylation to yield the key alcohol intermediate (16a). The alcohol (16a) was then phosphorylated to yield the target (2S)-compounds 19a and 20a via formation of 17a and 18a intermediates, using the similar chemistry as in Scheme 1. From O-benzyl-Boc-(D)-serine as starting material (2S)-analogs 19b and 20b were synthesized. All compounds were characterized by ¹H NMR, mass spectroscopy and, in case of final compounds, elemental analyses.

Previously reported DGPP 8:0 (1) and PA 8:0 (2) subtype selective antagonists of LPA₁ and LPA₃ receptors with an order of magnitude preference for LPA₃ receptor were derived from the natural sources and were available only in (R)-enantiomeric form. The activities of the (S)-enantiomers had not assessed at LPA GPCR. The inventors hypothesized that the PA 8:0 scaffold interacts with LPA receptors in a stereoselective manner, and synthesized and evaluated several PA 8:0 analogs. Keeping the hydrophobic chain length constant as in PA 8:0, they modified the phosphate headgroup to a thiophosphate, glycerol backbone to a serine, and varied the hydrophobic chain linkage, to provide analogs that were tested for the agonist and antagonist activities at LPA₁, LPA₂ and LPA₃ receptors.

RH7777 cells, which lack LPA GPCR, were stably transfected with individual LPA₁, LPA₂ and LPA₃ receptors and used for the in vitro screening. The ability of these compounds to activate intracellular LPA receptor PPARγ was also assessed in CV1 cells transfected with an acyl-coenzyme A oxidase-luciferase (PPRE-Acox-Rluc) reporter gene. The results obtained are shown in Table 5.

(2S)-PA 8:0 compound (7a), like its enantiomer (2R)-PA 8:0 (2), showed subtype selective antagonism at LPA₁ and LPA₃ receptors with no effect on LPA₂ receptor. PA analogs enantioselectively antagonize both LPA₁ and LPA₃ receptors with a moderate preference for S-isomer at LPA₁. The antagonistic selectivity is reversed at LPA₃ receptor, which showed a preference for (2R)-PA 8:0 over (2S)-isomer. (2R)-TPA 8:0 (8b) was a more potent antagonist than the phosphate analog (2R)-PA 8:0 (2) at both the LPA₁ and LPA₃ receptors, and a partial agonist of LPA₂. 8b was identified as the most potent and selective LPA₃ receptor antagonist reported so far with a Kᵢ value of 5 nM and 75-fold selectivity over LPA₁. (2S)-TPA 8:0 (8a) lacked LPA_{1/3} antagonism, but was a partial agonist at LPA₂ and LPA₃. Results for these newly-synthesized compounds, in accordance with previously published reports, show that LPA receptors exhibit stereoselectivity in interacting with the sn-2 substituted glycerol analogs.

To increase the stability of the acyl-PA 8:0 analogs against chemical as well as phospholipase A (PLA) degradation, the inventors synthesized 12a-b and 13a-b, alkyl derivatives of PA 8:0, and evaluated their agonist and antagonist properties at LPA GPCR. In general, thiophosphates were more potent than corresponding phosphates regardless of agonist/antagonist activity. Enantiospecific activation of LPA_{1/3} receptors by (2R)-APA 8:0 (12b) and 2(R)-ATPA 8:0 (13b) was observed. Compound 13b, which has the identical (R)-configuration as the endogenous LPA, was the most potent and LPA₃ subtype-selective receptor full agonist (EC₅₀=3 nM, Eₘₐₓ=109%), and was ˜230 and ˜1900 fold selective for LPA₃ over LPA₁ and LPA₂, respectively. At LPA₃ receptor, dioctyl thiophosphate analog 13b was a more potent agonist than the corresponding phosphate (12b) and LPA 18:1 (Table 5). In contrast to the (2R)-alkyl analogs, the opposite (2S)-enantiomers were antagonists at LPA_{1/3} receptors. Although compounds (R)-VPC12204 and (S)-VPC12249 were the first to demonstrate enantiospecific agonist and antagonist responses, respectively, at LPA, receptor, both enantiomers were antagonists at LPA₃ receptor. The enantiosepecific activation of LPA_{1/3} receptors by APA analogs may be due to the favorable orientation of the conformationally flexible alkyl side chains of (R)-isomers in ligand binding pocket of these receptors. The sidechains of acyl PA analogs are relatively constrained due to the ester linkage to glycerol, and may not be able to have these favorable interactions with the receptors. Except 2(R)-ATPA 8:0 (13b), which was a weak and partial agonist of LPA₂, alkyl PA analogs had no effect on LPA₂ receptor.

Replacement of the glycerol backbone by serine is well tolerated at LPA GPCR. Surprisingly, SDP 8:0 analogs (19a-b) were identified as LPA₃ receptor subtype-specific antagonists with no effect on LPA₁ and LPA₂ receptors. SDP 8:0 isomers also demonstrated enantioselectivty in LPA₃ antagonism with a preference for (S)-isomer (19a) over R (19b). Thiophosphate head group modification in serinediamides (20a-b) not only improved the LPA₃ antagonistic activity but also resulted in loss of LPA₃ subtype-specificity by rendering the LPA, antagonistic ability.

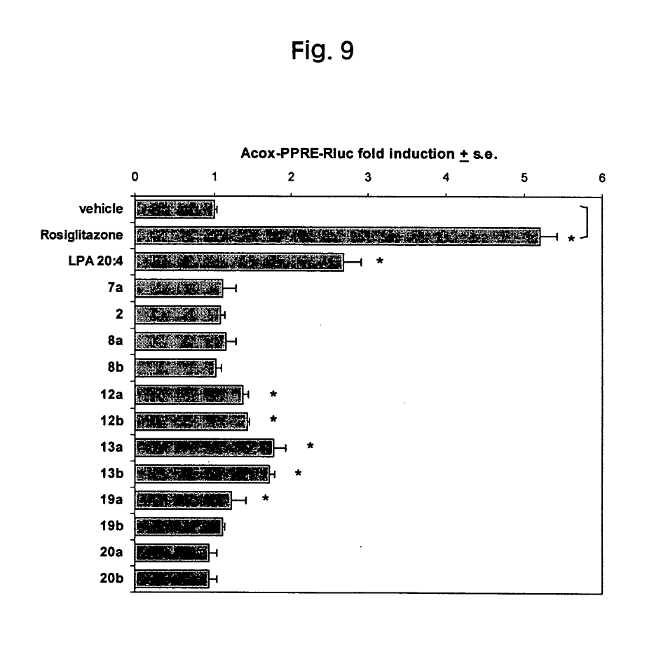

Results of in vitro PPARγ activation assay of these compounds in CV1 cells, transfected with PPARγ and PPRE-Acox-Rluc reporter gene, are shown in FIG. 9. Alkyl-PA analogs showed PPARγ activation, while PA and serinediamides were unable to activate PPARγ (FIG. 9). Unlike the enantiospecific responses compounds at LPA GPCR by APA analogs, there was no stereoselectivity observed in PPARγ activation by these analogs. Compound 19a is a selective LPA₃ antagonist with no effect on LPA_{1/2}, and also an agonist of PPARγ. But 19b retains the LPA₃ receptor selectivity and has no effect on PPARγ, making it a true LPA₃ receptor specific antagonist.

Compound 13b was identified as a potent LPA₃ receptor subtype-selective agonist and compound 8b (Kᵢ=5 nM) as the most potent subtype-selective LPA₃ receptor antagonist so far. Finally, using serine as a backbone substitute, an LPA₃ receptor specific antagonist 19b was discovered with no effect on LPA₁, LPA₂ and PPARγ.

TABLE 5
Effects of PA 8:0 analogs on LPA₁₋₃ transfected
RH7777 cells and activation of PPARγ

| | | | LPA₁ | | LPA₂ | | LPA₃ | |
|---|---|---|---|---|---|---|---|---|
| | | | EC₅₀ | IC₅₀ | EC₅₀ | IC₅₀ | EC₅₀ | IC₅₀ |
| | | | (Eₘₐₓ)^{a} | (Kᵢ) | (Eₘₐₓ) | (Kᵢ) | (Eₘₐₓ) | (Kᵢ) |
| Cpd | R/S | X | nM | nM | nM | nM | nM | nM |
| 7a | S | O | NE^{b} | 433 | NE | NE | NE | 207 |
| | | | | (221) | | | | (119) |
| 2 | R | O | NE | 692 | NE | NE | NE | 85 |
| | | | | (407) | | | | (39) |
| 8a | S | S | NE | NE | 7170 | NE | 115 | NE |
| | | | | | (17) | | (30) | |
| 8b | R | S | NE | 686 | 6330 | NE | NE | 11 |
| | | | | (360) | (58) | | | (5) |
| 12a | S | O | NE | 1580 | NE | NE | NE | 143 |
| | | | | (486) | | | | (50) |
| 12b | R | O | 3260 | NE | NE | NE | 164 | NE |
| | | | (57) | | | | (109) | |
| 13a | S | S | NE | 328 | NE | NE | NE | 184 |
| | | | | (139) | | | | (67) |
| 13b | R | S | 695 | NE | 5720 | NE | 3 | NE |
| | | | (30) | | (27) | | (109) | |
| 19a | S | O | NE | NE | NE | NE | NE | 414 |
| | | | | | | | | (196) |
| 19b | R | O | NE | NE | NE | NE | NE | 935 |
| | | | | | | | | (489) |
| 20a | S | S | NE | 476 | NE | NE | NE | 251 |
| | | | | (152) | | | | (117) |
| 20b | R | S | NE | 7390 | NE | NE | NE | 302 |
| | | | | (2850) | | | | (118) |

^{a}Eₘₐₓ = maximal efficacy of the drug/maximal efficacy of LPA 18:1, expressed as the percentage.
^{b}NE = no effect.

TABLE 6
Additional Analogs Prepared and Tested

| Compound | LPA1 | LPA2 | LPA3 |
|---|---|---|---|
| 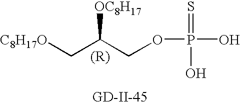 | EC₅₀: 695 nM (Partial agonist) | EC₅₀: 1.02 uM (Partial agonist) *transient | EC₅₀: 3 nM (Full agonist) |
| 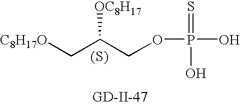 | IC₅₀: 328 nM (200 nM LPA) | Weak stimulation *transient | IC₅₀: 184 nM (200 nM LPA) |
| 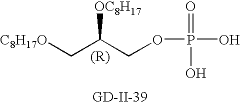 | EC₅₀: 3.26 uM (Partial agonist) | EC₅₀: 2.53 uM(?) (Partial agonist) *transient | EC₅₀: 164 nM (Full agonist) |
| 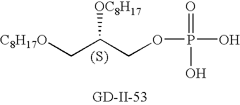 | IC₅₀: 1.58 uM (200 nM LPA) | Weak inhibition(?) *transient | IC₅₀: 143 nM (200 nM LPA) |

The invention therefore provides compounds comprising LPA analogs 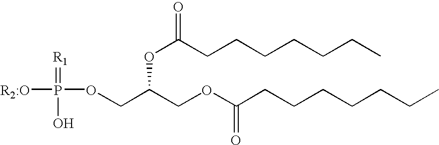
where R₁ is O or S and R₂ is a linear or branched chain, saturated or unsaturated, substituted or unsubstituted hydrocarbon having an agonist or antagonist effect on LPA receptor 1, 2, 3, or PPARγ. Compounds described by the present invention may also include, for example, salts, preferably water-soluble salts, such as ammonium, diammonium, potassium salts of the fatty alcohol phosphate.

As used herein, an “analog” is a chemical compound that is structurally or functionally similar to a known compound. Compounds of the present invention have demonstrated that they provide a benefit in modulating the effects of LPA through its extra-cellular receptors LPA₁, LPA₂, and/or LPA₃, as well as through its intracellular receptor PPARγ and the enzyme autotaxin. Compounds as described herein, or pharmaceutically acceptable salts of the compounds, may be administered to a subject separately or together in any conventional dosage forms, including, oral, buccal, sublingual, ocular, topical (e.g., transdermal), parenteral (e.g., intravenous, intramuscular, or subcutaneous), rectal, intracisternal, intravaginal, intraperitoneal, intravesical, local (e.g., powder, ointment, or drop), nasal and/or inhalation dosage forms. For therapeutic use, compounds of the present invention may be provided to a patient in oral form by means of tablets, capsules, liquids, softgels, or other modes of delivery known to those of skill in the art. Patients may also receive treatment using one or more compounds of the present invention delivered intravenously, intraperitoneally, intranasally, via a device to dispense medication at a steady rate, at predetermined intervals, as determined by the patient, or as the need for the medication is detected. Therapeutic medications comprising compounds pharmaceutically acceptable salts thereof may be administered in the form of a pharmaceutical composition comprising a pharmaceutically acceptable carrier, vehicle, or diluent.

As indicated herein, compounds have been described that have been determined to have effect at nanomolar concentrations. Some compounds demonstrate certain effects at millimolar concentrations. Determination of appropriate dosages for therapeutic use, given the information regarding effective concentrations provided herein, is within the skill of those in the art of pharmaceutical design and production.

The invention may be further described by means of the following non-limiting examples:

### EXAMPLES

Synthesis of Darmstoff Analogs

Bis-(2-cyano-ethyl)-2-heptadec-8-enyl-(1,3)dioxolan-4yl methyl phosphate (5). To a solution of alcohol 4 (0.245 g, 0.72 mmol) in dichloromethane (15 ml), 1H-tetrazole (0.2 g, 2.85 mmol) was added. After 10 minutes, to this solution biscyano ethyl diisopropyl phosphoramidite (0.39 g, 1.44 mmol) was added and stirred for 30 minutes. H₂O₂ (0.25 ml) was added to the reaction mixture and stirred for an additional 30 minutes. The reaction mixture was diluted with dichloromethane (100 ml) and the solution was washed sequentially with saturated aqueous Na₂S₂O₅, saturated NaHCO₃, water, brine and dried over Na₂SO₄. Solvent was removed in vacuo and the residue was purified by column chromatography (silica gel, acetone: hexanes) to give 0.3 g (80%) of 5. ¹HNMR (CDCL₃, 300 mHz) δ 0.89 (t, J=6.6 Hz, 3H), 1.29 (m. 24H), 2.03 (M, 4H), 2.82 (m, 4H), 3.57-3.68 (m, 1H), 3.84-3.96 (m, 1H), 4.1-4.2 (m, 2H), 4.25-4.45 (m, 5H), 4.57 (t, J=5.1 Hz, 0.5H), 4.88-5.0 (dt, J=4.8 Hz, 0.5H), 5.35 (m, 2H); ESIMS m/z 549.5 (M⁺+23).

2-Heptadec-8-enyl-(1,3)dioxolan-4yl methyl dipotassium phosphate (6). A solution of 5 (0.5 g, 0.095 mmol) in methanolic KOH (1M, 2 ml) was stirred for 3 hours, concentrated in vacuo, and the residue was dissolved in water and passed through a sep-pak syringe cartridge (C18). Eluted with methanol, fractions containing product were pooled and solvent was removed in vacuo to give 6 (0.045 g, 85%) as amorphous powder. ¹HNMR (CD₃OD, 300 MHz) δ 0.86 (m, 3H), 1.27 (m, 24H), 2.0 (m, 4H), 3.77-4.0 (m, 3H), 4.14-4.21 (m, 1H), 4.26-4.35 (m, 1H), 4.5-4.66 (m, 1H), 4.89 (t, J=4.8 Hz, 0.5H), 5.0 (t, J=4.8 Hz, 0.5H), 5.35 (, 2H); ESIMS m/z 419.5 (M⁺−1).

Bis-(2-cyano-ethyl)-2-heptadec-8-enyl-(1,3)dioxolan-4yl methyl thiophosphate (7). To a solution of alcohol, 4 (0.274 g, 0.80 mmol) in dichloromethane (15 ml), 1H-tetrazole (0.17 g, 2.4 mmol) was added. After 10 minutes, to this solution biscyano ethyl diisopropyl phospharamidite (0.44 g, 1.66 mmol) was added and stirred for 30 minutes. Sulfur powder (0.076 g, 2.4 mmol) was added to the reaction mixture and refluxed for 2 h. The mixture was then cooled to RT, concentrated in vacuo and the residue was purified by column chromatography (silica gel, acetone:hexanes) to give 0.32 g (73%) of 7. ¹HNMR (CDCl₃, 300 MHz) δ 0.86 (t, J=6.9 Hz, 3H), 1.29 (brs, 24H), 2.03 (m, 4H), 2.69-2.86 (m, 4H), 3.6-4.0 (m, 4H), 4.1-4.4 (m, 5H), 4.56 (t, J=4.8 Hz, 0.35H), 4.88-5.0 (dt, J=4.5 Hz, 0.65H), 5.35 (m, 2H).

2-Heptadec-8-enyl-(1,3)dioxolan-4yl methyldipotassium thiophosphate (8). Compound 8 was prepared by the same procedure as that of 6 as amorphous powder (0.058 g, 88%). ¹HNMR (D₂O, 300 MHz), δ 0.78 (brs, 3H), 1.19 (brs, 24H), 1.93 (m, 4H), 3.76-3.85 (m, 3H), 4.05-4.25 (m, 2H), 4.4-4.47 (m, 0.4H), 4.75 (m, 0.75H) 4.97 (m, 0.3H), 5.25 (m, 2H).

TABLE 6
Summary of Testing of the Acetal Phosphate Analogs on the Calcium Mobilization Response

| Compound | RH-LPA₁ cells | RH-LPA₂ cells | PC3 cells |
|---|---|---|---|
| 13:0 acetal | Inhibited 200 nM LPA response with | Inhibited 200 nM LPA response with | Inhibited 100 nM LPA response with |
| phosphate | an IC₅₀ of 1.11 μM and 300 | an IC₅₀ of 397 nM and 300 | an IC₅₀ of 1.86 μM and 300 |
| | nM LPA with an IC₅₀ of 2.15 μM | nM LPA with an IC₅₀ of 657 nM | nM LPA with an IC₅₀ of 2.11 μM |
| 18:1 Δ 9 acetal | Inhibited 200 nM LPA response with | Inhibited 200 nM LPA response with | Inhibited 200 nM LPA response with |
| phosphate | an IC₅₀ of 915 nM and 300 | an IC₅₀ of 460 nM and 300 | an IC₅₀ of 1.04 μM and 300 |
| | nM LPA with an IC₅₀ of 1.24 μM | nM LPA with an IC₅₀ of 777 nM | nM LPA with an IC₅₀ of 1.5 μM |
| 18:1 Δ 9 acetal | Alone, the compound had a | Alone, the compound had a | Alone, the compound had a |
| thiophosphate | stimulatory effect. Its maximal | stimulatory effect. Its maximal | stimulatory effect. Its maximal |
| | response was about 50% of the LPA- | response was about 74% of the LPA- | response was about 86% of the LPA- |
| | alone max. response. The | alone max. response. The | alone max. response. The |
| | thiophosphate had an EC₅₀ of 2.17 μM. | thiophosphate had an EC₅₀ of 639 nM. | thiophosphate had an EC₅₀ of 1.37 μM. |
| | Co-administration of the compound | Co-administration of the compound | Co-administration of the compound |
| | with LPA synthetized the LPA | with LPA synthetized the LPA | with LPA synthetized the LPA |
| | response. | response. | response. |

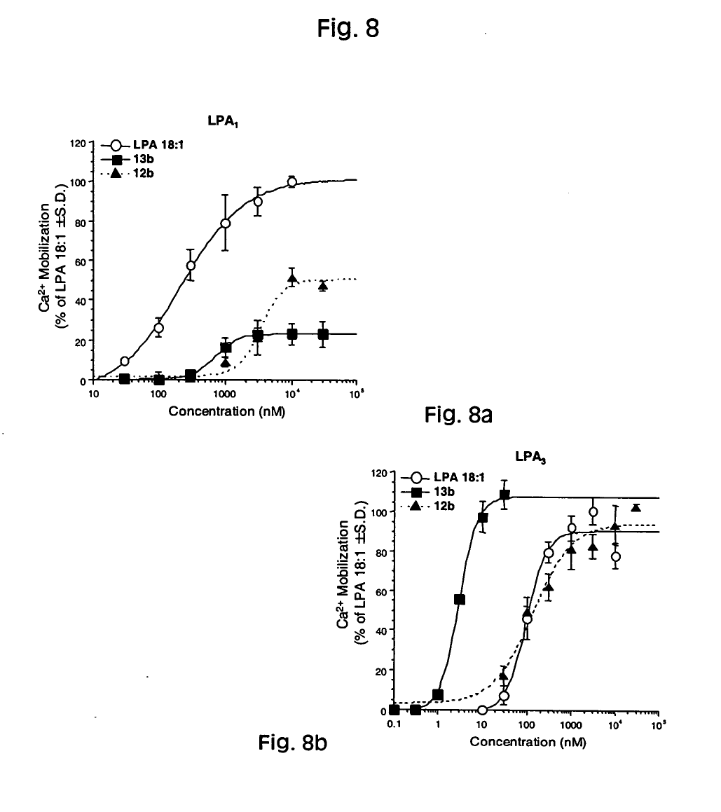

FIG. 8 graphs dose-response relationships for LPA 18:1, 12b and 13b in RH7777 cells expressing LPA, (FIG. 9a) and LPA₃ (FIG. 9b). Intracellular Ca²⁺ transients were measured in response to the application of increasing concentrations of compounds 12b and 13b and compared to transients elicited by LPA 18:1. Data points represent the average of four measurements. (2R) Alkyl PA analogs (12b and 13b) are agonists at LPA₁ and LPA₃ receptors expressed in RH7777 cells.

What is claimed is:

1. An LPA analog that acts as an agonist or antagonist of LPA₁, LPA₁, LPA₃, PPARγ, and/or autotaxin, the LPA analog comprising an acetal phosphatidic acid.

2. An LPA analog as described by claim 1 wherein the analog comprises 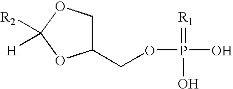
where R₁ is O or S, and R₂ is a straight or branched chain saturated or unsaturated, linear or cyclic hydrocarbon, substituted or unsubstituted, preferably C6 to C24, having an agonist or antagonist effect on LPA₁, LPA₂, LPA₃, PPARγ, and/or autotaxin.

3. An LPA analog as in claim 1 wherein the analog comprises a water soluble salt of an acetal phosphatidic acid.

4. A therapeutic method of modulating an LPA-, PPARγ-, and/or autotaxin-mediated disease in a human or animal subject, the method comprising administering to the subject a therapeutically effective amount of an acetal phosphatidic acid having agonist or antagonist activity on LPA₁, LPA₁, LPA₃, PPARγ, autotaxin, or a combination thereof.
